(19) United States
(12) Patent Application Publication
(10) Pub. No.: US 2002/0150553 A1
(43) Pub. Date: Oct. 17, 2002

(54) Compositions containing C-terminal polypeptides of angiogenic chemokines and methods of use

(75) Inventor: Maria Manuela Marques Martins-Green, Riverside, CA (US)

Correspondence Address: LAW OFFICES OF JONATHAN ALAN QUINE, P O BOX 458, ALAMEDA, CA, 94501

(73) Assignee: The Regents of the University of California, Oakland, CA

(21) Appl. No.: 10/121,119

(22) Filed: Apr. 10, 2002

Related U.S. Application Data
(62) Division of application No. 09/429,050, filed on Oct. 29, 1999.

(51) Int. Cl. A61K 38/19

(52) U.S. Cl. 424/85.1

(57) ABSTRACT

Compositions containing the C-terminal region of cCAF or IL-8 can be used in methods for inducing an angiogenic response, without attracting leukocytes, when administered to an animal or human subject in an amount sufficient to stimulate sprouting of new blood vessels.

### FIELD OF THE INVENTION

The present invention relates generally to the field of angiogenesis. More particularly it concerns compositions containing angiogenic polypeptides and methods of using such angiogenic polypeptides to induce an angiogenic response.

### BACKGROUND OF THE INVENTION

Angiogenesis is the process by which new blood vessels develop from pre-existing ones by endothelial cell proliferation and migration. It occurs during embryonic development and in adults during ovulation, placentation, menstruation, and wound healing. In all of these cases, angiogenesis is tightly regulated and in the latter four cases it is also of short duration. However, persistent blood vessel development is often found in association with diseases such as diabetic retinopathy, glaucoma and rheumatoid arthritis, as well as with a variety of tumours. Therefore, it is of great importance to determine the molecular mechanisms that trigger blood vessel sprouting, growth, and invasion of tissues, so that potential therapeutic agents can be developed.

A variety of cytokines are known to modulate angiogenesis. Within this group of molecules a number of small molecular weight proteins that belong to the chemokine superfamily [Taub D D, Oppenheim J J (1994) Chemokines, inflammation and the immune system. Therapeutic Immunol 1:229-246; Baggiolini M, Dewald B, Moser B (1994) Interleukin-8 and related chemotactic cytokines-CXC and CC chemokines. Adv Immunol 55:97-179; Prieschl E E, Kulmburg P A, Baumruker T (1995) The nomenclature of chemokines. Internatl Archives Allergy Immunol 107:475-483.] also play important roles in the development of blood vessels [e.g. Wilmer J L, Burleson F G, Kayama F, Kanno J, Luster M I (1994) Cytokine induction in human epidermal keratinocytes exposed to contact irritants and its relation to chemical-induced inflammation in mouse skin. J Invest Dermatol 102:915-922; Strieter R M, Polverini P J, Arenberg D A, Kundel S L (1995a) The role of CXC chemokines as regulators of angiogenesis. Shock 4:155-160; Nanney L B, Mueller S G, Bueno R, Peiper S C, Richmond A (1995) Distribution of melanoma growth stiinulatory activity or growth-regulated gene and the interleukin-8 receptor B in human wound repair. Am J Pathol 147:1248-1260; Martins-Green M, Hanafusa H (1997) The 9E3/CEF4 gene and its product the chicken Chemotactic and Angiogenic Factor (cCAF): potential roles in wound healing and tumor development. Cytokines Growth Factors 8(3):219-230]. Because of their chemotactic properties, these small cytokines are called chemokines; they are secreted proteins with molecular masses of 6-20 kDa [Taub D D et al.; Baggiolini et al. (1994); Sugano S, Stoeckle M Y, Hanafusa H (1987) Transformation by Rous sarcoma virus induces a novel gene with homology to a mitogenic platelet protein. Cell 49:321-328; Bedard P-A, Alcorta D, Simmons D L, Luk K -C, Erikson R L (1987) Constitutive expression of a gene encoding a polypeptide homologous to biologically active human platelet protein in Rous sarcoma virus-transformed fibroblasts. Proc Natl Acad Sci USA 84:6715-6719; Yoshimura T, Matsushima K, Tanaka S, Robinson E A, Appella E, Oppenheirn J J, Leonard E J (1987) Purification of human monocyte-derived neutrophil chemotactic factor that has peptide sequence similarity to other host defense cytokines. Proc Natl Acad Sci USA 84:9233-9237; Richmond A, Thomas H J (1988) Melanoma growth stimulatory activity: Isolation from human melanoma tumors and characterization of tissue distribution. J Cell Biochem 36:185-198; Rollins B J, Stiles C D (1989) Serum-inducible genes. Adv Cancer Res 53:1-32; Derynck R, Balentien E, Han J H, Thomas H G, Wen D, Samantha A K, Zachariae C O, Griffin P R, Brachmann R, Wong W L, Matsushima K, Richmond A (1990) Recombinant expression, biochemical characterization, and biological activities of the human MGSA/gro protein. Biochemistry 29:10225-10233; Martins-Green M, Aotaki-Keen A, Hjelmeland L, Bissell M J (1992) The 9E3 protein: immunolocalization in vivo and evidence for multiple forms in culture. J Cell Sci 101:701-707; Baggiolini M, Dewald B, Moser B (1997) Human chemokines: an update. Annual Review of Immunology 15:675-705; and Adams D H, Loyd A R (1997) Chemokines: leukocyte recruitment and activation cytokines. Lancet 349:490-495] that are encoded by immediate early response genes and are evolutionarily conserved. [Baggiolini et al. (1994); Stoeckle M Y, Barker K A (1990) Two burgeoning families of platelet factor 4-related proteins: Mediators of the inflammatory response. The New Biologist 2:313-323; Sager R, Haskill S, Anisowicz A, Trask D, Pike M C (1991) GRO: a novel chemotactic cytokine. Adv Exp Med Biol 305:73-77; Sherry B, Cerami A (1991) Small cytokine superfamily. Curr Opin Immunol 3:56-60; Mukaida N, Matsushima K (1992) Regulation of IL-8 production and the characteristics of the receptors for I L-8. Cytokine 4:41-53; Oppenheim J J (1993) Overview of chemokines. Adv Exp Med Biol 351:183-186; and Sozzani S, Locati M, Allavena P, Van Damme J, Mantovani A (1996) Chemokines: a superfamily of chemotactic cytokines. J Clin Lab Res 26:69-82.]

Chemokines have four conserved cysteines that form two disulfide bonds which anchor the tertiary structure of the molecules and are important in their function. [Stoeckle M Y, Barker K A (1990) Two burgeoning families of platelet factor 4-related proteins: Mediators of the inflammatory response. The New Biologist 2:313-323; Clore G M, Appella E, Yamada M, Matsushima K. Gronenborn A M (1990) Three-dimensional structure of interleukin 8 in solution. Biochemistry 1-9:1689-1693; Baldwin E T. Weber I T, St. Charles R, Xuan J -C, Appella E, Yamada M, Matsushima K, Edwards B F P, Clore G M, Gronenborn A M, Wlodawer A (1991) Crystal structure of interleukin 8: symbiosis of NMR and crystallography. Proc Natl Acad Sci USA 88:502-506; Fairbrother W J, Reilly D, Colby T J, Hesselgesser J, Horuk R (1994) The solution structure of melanoma growth stimulating activity. J Mol Biol 242:252-270; Fairbrother W J, Shelton N J (1996) Three-dimensional structures of the chemokine family in Horuk R (ed.) Chemoattractant Ligands and Their Receptors, 55-86, New York: CRC Press; Clark-Lewis I, Kim K -S, Rajarathnam K, Gong J -H, Dewald B, Moser B (1995) Structure-activity of chemokines, J Leukocyte Biol 57:703-711; Clore G M, Gronenborn A M (1995) Three-dimensional structure of alpha and beta chemokines. In Horuk R (ed.) Chemoattractant Ligands and Their Receptors. New York: CRC Press; Gupta S K, Hassel T, Singh J P (1995) A potent inhibitor of endothelial cell proliferation is generated by proteolytic cleavage of the chemokine platelet factor 4. Proc Natl Acad Sci USA 92:7799-7803; Osterman D G, Griffin G L, Senior R M, Kaiser E T, Deuel T F (1982) The carboxy-terminal tridecapeptide of platelet factor 4 is a potent chemotactic agent for monocytes. Biochem Biophys Res Commun 107:130-135; Zucker M R, Katz I R, Thorbecke G J, Milot D C, Holt J (1989) Immunoregulatory activity of peptide related to platelet factor-4. Proc Natl Acad Sci USA 86:7571-7574; Maione T E, Gray S G, Petro J, Hunt A, Donner A L, Bauer S I, Carson H F, Sharpe R J (1990) Inhibition of angiogenesis by recombinant human platelet factor-4 and related peptides. Science 247:77-79; Martins-Green M, Stoeckle M, Wimberly S, Hampe A, Hanafusa H (1996) The 9E3/CEF4 cytokine: kinetics of secretion, processing by plasmin, and interaction with extracellular matrix. Cytokine 8:448-459; Martins-Green M, Bissell M J (1990) Localization of 9E3/CEF-4 in avian tissues. Expression is absent in Rous sarcoma virus-induced tumors but is stimulated by injury. J Cell Biol 110:581-595; Vaingankar S, Martins-Green M (1997) Thrombin activation of the 9E3/CEF4 chemokine involves tyrosine kinases including c-src and the EGF receptor. J Biol Chem 273:5226-5234; and Clark R A F, Henson P M (1996) (eds) The Molecular and Cellular Biology of Wound Repair. New York: Plenum Press] Structurally, they consist of a short, flexible, N-terminus followed by a loop, three β-pleated sheets and a C-terminal α-helix, all of which may represent functional domains [Fairbrother et al. (1996); Clark-Lewis et al. (1995); and Clore et al. (1995)]. Over the years investigators have come to realize that chemokines as well as other cytokines perform multiple functions. For a long time this discovery was perplexing. However the subsequent realization that chemokines display multiple forms and/or multiple active domains offered the satisfying explanation that this diversity might be responsible for the different functions. A good example of this functional diversity is PF4. The whole PF4 molecule minus the N-terminal 16 aas inhibits mitogenesis of endothelial cells in culture (Gupta et al.), whereas the C-terminus tridecapeptide has chemotactic and immunoregulatory properties (Osterman et al.; and Zucker et al.) and is angiostatic in the chorioallantoic membrane (CAM) assay (Maione et al.).

The chemokine superfamily is subdivided into families based on the position of the first two cysteines. One of the major families, called the C—X—C or α family, has the first two cysteines separated by a single amino acid. Chicken chemotactic and angiogenic factor (cCAF), the product of the 9E3 gene, belongs to this family along with human chemokines such as interleukin 8 (IL-8), MGSA/groα, IP-10, PF-4 and β-thromboglobulin (β-TG) (see Stoeckle et al.). cCAF is secreted as a 9 kDa protein [Martins-Green et al. (1996)] that is highly homologous to human IL-8 (51%), MGSA/groα (45%) and β-thromboglobulin (43%). After secretion, cCAF can be processed to a smaller form (˜7 kDa) by cleavage of the N-terminus, and this form is primarily found in association with specific extracellular matrix molecules [Martins-Green et al. (1996)]. The 9E3 gene is expressed abundantly shortly after wounding in vivo, the expression persists at lower but still elevated level during formation of the granulation tissue, and the levels of expression in the granulation tissue decrease with distance from the site of the wound. Thrombin, which is released from the blood immediately upon wounding, is the most potent natural stimulator of 9E3 expression and of cCAF production, suggesting that this enzyme might be the stimulator of the 9E3 gene shortly after wounding (Vaingankar et al.). In addition, plasmin, an enzyme that appears at the wound site during the inflammatory phase of healing (see Clark et al.), cleaves cCAF at the N-terminus (both in culture and in the test tube) to the smaller form that is capable of binding to matrix molecules present in the granulation tissue. This binding to the matrix could potentially be responsible for the gradient observed in vivo [Martins-Green et al. (1996)].

The full 9-kDa form of cCAF is chemotactic for leukocytes and also is angiogenic. At low concentrations, it is chemotactic for monocyte/macrophages and lymphocytes and it causes oriented blood vessel growth. At higher concentrations, cCAF is not chemotactic for leukocytes, nor does it cause oriented blood vessel growth, but rather causes tortuosity and sprouting of young blood vessels (Martins-Green et al., 1998). In addition, cCAF triggers a cascade of events that leads to hyperproliferation of keratinocytes and formation of granulation-like tissue with deposition of ECM, strongly suggesting a role for cCAF in these events of wound healing.

It has been determined that cCAF and closely related CXC-chemokines, such as IL-8, exhibit both chemotactic and angiogenic functions. However, the contribution of the various structural domains of the chemokine molecules to these biological activities have not been clearly defined. Since the involvement of CXC-chemokines in angiogenesis is relevant to tumor development and wound healing, a more precise understanding of the structural elements that control these biological activities is needed.

### SUMMARY OF THE INVENTION

The present invention fulfills this need by showing that a C-terminal peptide of the CXC-chemokine cCAF reproduces angiogenic properties of the full molecule, such as chemotaxis for blood vessels and vessel sprouting. However the C-terminal peptide is devoid of chemotactic activity for leukocytes and does not lead to granulation-like tissue formation.

Accordingly, the present invention provides for an angiogenic composition that contains an effective amount of an angiogenic polypeptide in a suitable carrier. Compositions containing an effective amount of the angiogenice polypeptide can induce tortuosity and sprouting of new blood vessels. The angiogenic polypeptide is a C-terminal fragment of a CXC chemokine devoid of chemotactic activity for leukocytes. The angiogenic polypeptide includes an amino acid sequence substantially equivalent to a C-terminal alpha helical domain of a wild-type CXC chemokine. The wild-type chemokine sequence from which the angiogenic polypeptide is derived typically contains four conserved cysteine residues, an ELR motif and a WVQ motif. The C-terminal alpha helical domain included in the angiogenic polypeptide comprises at least the WVQ motif and continues C-terminally at least about twelve residues beyond the WVQ motif of the wild-type chemokine sequence. Moreover, the N-terminus of the polypeptide typically begins at a residue downstream from the ELR motif. Examples of the C-terminal alpha helical domains of the present invention include amino acids 83 to 97 of cCAF (SEQ ID NO:1) or human interleukin 8 (SEQ ID NO: 3). A most preferred angiogenic polypeptide is a 28 amino acid peptide of cCAF corresponding to amino acid residues 76 to 103 of SEQ ID NO:1.

The compositions of the present invention can be used in methods for inducing angiogenesis. Generally, the angiogenic composition is administered to an animal or a human subject in a biologically effective amount that induces sprouting of new blood vessels.

Another version of the present invention is a method to improve wound healing, where an effective amount of the angiogenic composition is administered in proximity to a wound.

Yet another version of the present invention is a method where localized administration of an effective amount of the angiogenic composition to a tissue increases the density of microvessels in the tissue.

In addition to such beneficial uses, the methods and compositions of the present invention are useful in bioassays for screening angiogenic or angiostatic agents.

### BRIEF DESCRIPTION OF THE DRAWINGS

These and other features, aspects, and advantages of the present invention will become better understood with regard to the following description, appended claims, and accompanying drawings, where:

FIG. 1 shows the time-dependent release of the C-terminus peptide from methylcellulose pellets; Five microliters of 0.5%, methylcellulose containing 300 ng of ¹²⁵I-labeled C-terminal peptide were placed in the bottom of Eppendorf tubes, allowed to dry and then covered with 300 μl of sterile PBS containing 0.02% sodium azide and incubated at 37° C. Samples of 280 μl of saline were removed periodically for scintillation counting and replaced by fresh saline. Error bars represent the standard deviation for 15 samples taken at each time point.

FIG. 2 shows the angiogenic effect of the C-terminal peptide in the CAM; Angiogenesis in the CAM was evaluated using 2 mm diameter, 0.5% methylcellulose pellets (p) containing various treatments. (A) Natural CAM at 10 days of development shows dendritic pattern with hierarchical sizes of blood vessels, denoted by primary [1], secondary [2], and tertiary [3]; (B) pellet with 100 ng BSA (negative control) shows no substantative difference from natural CAM; (C) pellet with 100 ng 9E3 peptide shows strong radial orientation of the secondary and tertiary blood vessels; (D) pellet with 100 ng peptide preincubated with excess antipeptide antibody shows some disturbance of the blood vessel pattern but no discernible radial pattern or increased numbers of vessels; (E) pellet with 500 ng bFGF (positive control) showed very similar effects to those caused by equivalent nanomolar concentration (100 ng) of the C-terminal peptide of cCAF (C). White spots on C and E are reflections. Scale bar=1000 μm.

FIG. 3 shows cross sections through a natural CAM and a CAM containing a pellet with the C-terminus peptide; (A) CAM with pellet (p) containing 100 ng of C-terminus peptide for 2 days showing only nominal differences from the control (C). The cells inside the large blood vessel are erythrocytes (these chicken cells are nucleated). (B) CAM treated for 2 days with pellet (p) containing 100 ng of the whole cCAF molecule showed the presence of leukocytes (primarily monocyte/macrophages, a few indicated by arrowheads). (C) Natural CAM showing sparse cells and a single blood vessel. Scale bars=100 μm.

FIG. 4 shows quantitation of CAMs with induced angiogenesis; Pellets containing BSA (□) (negative control) showed virtually no disturbance of CAM structure whereas pellets containing bFGF (▪) (positive control) showed angiogenesis 100% of the time. Stimulation of angiogenesis by pellets containing either the reduced (///) or non-reduced (\\\) cCAF peptide was virtually 100%, but when the peptide was preincubated with its antibody (XX), no angiogenesis was seen.

FIG. 5 shows dose dependence of the peptide angiogenic potential; Vessels were designated primary, secondary, and tertiary as described in FIG. 2. (A) Orientation of secondary and tertiary vessels plotted in terms of percentage of vessels of each category directed towards the pellet. Lower concentrations of the peptide (100 ng) are more effective in eliciting reorientation of secondary blood vessels towards the pellet whereas the higher concentrations (300 ng) are more effective in attracting tertiary vessels. (□), BSA; (▪), 500 ng bFGF; (///),100 ng C-terminal peptide; (\\\), 200 ng C-terminal peptide; (XX), 300 ng C-terminal peptide; (≡), C-terminal peptide+anti-C-terminal peptide antibody. Application of the Student t-test to these data (asterisks) showed statistically significant differences between the values for both secondary vessels (P=0.019) and tertiary vessels (P=0.030). (B) Blood vessel orientation grouped by type/concentration of agent and plotted as percentage of vessels toward the pellet minus percentage away. Distributions oriented toward the pellet plot as positive values; those directed away from the pellet, yield negative values. Equimolar amounts of peptide (C-terminus=100 ng) and bFGF (bFGF=500 ng) show indistinguishable effects. (□), secondary vessels: (▪), tertiary vessels.

FIG. 6 shows angiogenic properties of higher doses of the cCAF C-terminal peptide; (A) Pellets (p) containing 600 ng of peptide stimulated less blood vessel reorientation than lower doses (see FIG. 2 for comparison). (B) Higher magnification of another CAM treated the same as that in A, showing that the peptide induced tortuosity to many of the smaller vessels and abundant blood vessel sprouting (a few examples of small sprouts are indicated by arrowheads). (C) Natural CAM showing the normal morphology and number of tertiary blood vessels. (D) provides a quantitation of the sprouting effects of 1000 ng/pellet of the C-terminus peptide. A sprout was defined as having a length of one third or less than the tertiary branches of the normal CAM. CAMs treated with 1000 ng of C-terminus peptide showed about 10 times more sprouting than those treated with BSA at the same concentration. Scale bars=500 μm.

FIG. 7 shows the angiogenic effect of the C-terminal peptide in the wing web of a newly hatched chick. Hematoxylin and eosin stained cross sections of wing muscle tissue showing organization of the muscle fibers. (A) Very few blood vessels (arrowheads) can be seen amongst the untreated muscle fibers. A large nerve bundle can be seen (black dot) associated with three small blood vessels. (B) Similar section taken from the wing of a bird that has been treated with the C-terminus peptide. Note the presence of numerous small blood vessels (arrowheads) amongst the muscle fibers. (C) Similar section from a bird treated with only buffer vehicle for the peptide. The number of blood vessels (arrowheads) is similar to that of the control. Scale bar=50 μm.

### DETAILED DESCRIPTION OF THE INVENTION

The present invention stems from the inventor's discovery that the C-terminus of certain CXC chemokines, such as cCAF and IL-8, have angiogenic properties, but do not attract leukocytes. Accordingly, the present invention defines an important functional domain of cCAF and IL-8 that correlates with certain structural features, most notably the C-termnal alpha helical regions of the full length proteins. Furthermore, the present invention discloses compositions containing angiogenic polypeptides comprised of these important functional and structural domains. The compositions are useful in methods for inducing an angiogenic response, with important applications in wound healing and bioassays for angiogenic or angiostatic agents.

I. CXC Chemokines

Chemokines are secreted proteins that are evolutionarily conserved, are encoded by early response genes and are the principal chemoattractants for leukocytes. These proteins have four conserved cyteines that form two disulfide bonds, which anchor the tertiary structure of these molecules and are important for their function. Their molecular weights range between 6 k and 20 k, but multiple forms are frequently observed and appear to be related to activation of the molecule and/or to multiple functions.

The superfamily is subdivided into based on the position of the first two cysteines—in the CXC superfamily (also called the α family), the cysteines are separated by a single amino acid and in the CC family (also called the β family), the cysteines are adjacent. Recently, two new families have found, the C (also called the γ family) in which the first cysteine is missing and the CX₃C family in which the first two cysteines are separated by any three amino acids.

The angiogenic polypeptides of the present invention are derived from members of the CXC family. As used herein, the term CXC chemokine is used to refer to a cytokine that has the amino acid motif Cys Xaa Cys located in the N-terminal region, or a derivative or mutant thereof. Examples of CXC chemokines include cCAF/9E3/CEF4, IL-8/NAP1, MGSA/groα, ENA-78, GCP-2, MIP-2β/groβ, MIP-2β/groγ, CTAP-III, NAP-2, βTG, IP-10, MIG and PF4. For simplicity, the term “CXC chemokine” is used to describe both the native or wild type chemokines and those CXC chemokines with sequences altered by the hand of man (engineered chemokines).

More particluarly, the angiogenic polypeptides are derived from members of the ELR-CXC chemokine family. A CXC chemokine that contains the amino acid motif ELR is referred to as an “ELR-CXC chemokine (ELR-CXC),” whereas a CXC chemokine that does not contain this motif is termed and “XXX-CXC chemokine” or referred to as XXX-CXC. Examples of ELR-CXC chemokines include cCAF, IL-8, ENA-78, GCP-2, gro-α, -β and -γ, CTAP-III, NAP-2 and βTG. Examples of XXX-CXC chemokines include IP-10, MIG and PF4.

The term “wild type” refers to those CXC chemokines that have an amino acid sequence as found in the natural environment. This term therefore refers to the sequence characteristics, irrespective of whether the actual molecule is purified from natural sources, synthesized in vitro, or obtained following recombinant expression of a CXC chemokine-encoding DNA molecule in a host cell.

The terms “mutant, variant or engineered” CXC chemokine refer to those CXC chemokines the amino acid sequence of which have been altered with respect to the sequence of the chemokine found in nature. This term thus describes CXC chemokines that have been altered by the hand of man, irrespective of the manner of making the modification, e.g., whether recombinant DNA techniques or protein chemical modifications are employed.

“Native” CXC chemokines are those that have been purified from their natural sources, such as from tissues or from cultured, but otherwise unaltered, cells. Native CXC chemokines will also generally have wild type sequences.

“Recombinant” CXC chemokines are those molecules produced following expression of a CXC chemokine recombinant DNA molecule, or gene, in a prokaryotic or eukaryotic host cell, or even following translation of an RNA molecule in and in vitro translation system. “Synthetic” CXC chemokines are those chemokines produced using synthetic chemistry, most usually in the form of automated peptide synthesis. Both recombinant and synthetic CXC chemokines may have either wild type or mutant sequences, as designed.

The angiogenic compositions of the present invention are derived from ELR-CXC chemokines having angiogenic properties. Examples of ELR-CXC chemokines having angiogenic properties include cCAF and IL-8. cCAF is an avian chemokine that is more homologous to human IL-8 than any other chemokine, human or otherwise. It is also highly homologous to melanoma growth stimulating activity (MGSA) and to β-thromboglobulin (43%), two other human ELR-CXC chemokines having roles in wound healing, but whose angiogenic properties are uncertain.

II. Structural Features

The first of the CXC chemokines for which the tertiary structure has been determined, using NMR spectroscopy and X-ray crystallography is IL-8. The very high homology amongst these chemokines allows IL-8 to serve as a prototype for structure determination of other CXC chemokines. The structure of IL-8 includes a short, flexible N-terminus followed by a loop, three β-pleated sheets and a C-termal α-helix, all of which may represent funtional domains.

To model the the cCAF structure, we obtained the three dimensional structure of IL-8 from the PDB database and modified it by replacing the amino acids of IL-8 with those of cCAF using the Builder module of the Insight software (BIOSYM, San Diego, Calif.). The cCAF structure was subsequently optimized through energy minimization and molecular dynamics calculations using the Discover module (BIOSYM, San Diego, Calif.). After tens of thousands of iterations, we obtained a three-dimensionsal structure for cCAF.

The cCAF structure includes the same domains as IL-8, with the exception that the N-terminus of the molecule is much longer than that found in IL-8 and other CXC chemokines. The body of the molecule is very similar to the IL-8 structure with the loops and the β-pleated sheets in the same configuration. However, cCAF has assumed a more compact conformation than IL-8, with the C-terminal α-helix bending over the body of the molecule. The last five amino acids of the C-terminus, which are not present in IL-8, extend from the α-helix in a coiled configuration and align with the β-pleated sheets. The other major difference between the two molecules occurs at the N-terminus, where cCAF has 10 extra amino acids that are not present in the shorter forms of IL-8. The five most N-terminal amino acids assume an α-helix conformation, which bends towards the body of the molecule and lies very close to the C-terminus α-helix.

The alignment and surface exposure of the N-terminal and C-terminal α-helices of cCAF point to these domains as probable sites for intermolecular interactions. Moreover, the C-terminal a-helix has a very hydrophobic region, which could serve as a binding region to other molecules on cell-surface receptors.

The amino acid sequence of the cCAF pre-cursor is disclosed herein as SEQ ID NO: 1 and the cDNA encoding this sequence is SEQ ID NO:2. Similarly, the amino acid sequence of human IL-8 precursor is SEQ ID NO:3 and the cDNA sequence encoding human IL-8 is SEQ ID NO:4. For purposes of comparison, the amino acid and cDNA sequences encoding MGSA are SEQ ID NO:5 and SEQ ID NO:6, respectively.

TABLE 1

| | I1-8* | cCAF† | MGSA‡ |
|---|---|---|---|
| Signal peptide | 1-22 | 1-17 | 1-34 |
| Mature Form | 23-99 | 18-103 | 35-107 |
| ELR Motif | 31-33 | 30-32 | 40-42 |
| Disulphide bonds | 34, 61 | 33, 60 | 43, 69 |
| | 36, 77 | 35, 76 | 45, 85 |
| C-terminal helix | 83-97 | 82-97 | 92-102 |
| WVQ Motif | 84-86 | 83-85 | |

*aa residues from SEQ ID NO: 3
†aa residues from SEQ ID NO: 1
‡aa residues from SEQ ID NO: 5

Table 1 summarizes some of the structural features that are conserved among the ELR-CXC chemokines. These include the ELR motif found near the N-terminal end of the mature polypeptide; four conserved Cys residues, which form two disulphide bonds; and an alpha-helical region near the C-terminus of the molecules. The C-terminal helix of cCAF and IL-8 is about 15 to 16 amino acids long, whereas the C-terminal helix of MGSA is somewhat shorter at about 11 residues. Moreover the MGSA molecule lacks the WVQ motif shared by the C-terminal regions of cCAF and IL-8.

The present invention utilizes angiogenic polypeptides that contain a C-terminal alpha helical domain of a wild type CXC-chemokine, which has angiogenic properties. The angiogenic polypeptides will generally include at least about 15 amino acids of the C-terminal alpha helical regions of wild type cCAF or IL-8. Preferably, the polypeptides contain a WVQ motif, but do not include the ELR motif. For example, a polypeptide lacking a portion of the N-terminus of IL-8 or cCAF, including the ELR motif, but containing amino acid residues 83 to 97 of SEQ ID NO:3 or 82 to 97 of SEQ ID NO:1, i.e., the alpha helical domains of the IL-8 and cCAF molecules, would be considered angiogenic polypeptides according to the present invention. A most preferred version of the angiogenic polypeptide is the last 28 amino acid residues of cCAF.

The use of polypeptides having amino acid sequences that are substantially equivalent to the C-terminal alpha helical region of cCAF or IL-8 is also considered to be within the scope of the present invention. As used herein, IL-8 includes human, murine, bovine, pig, rabbit, sheep and other mammalian IL-8 molecules. Moreover, polypeptides containing sequences that are at least 70% identical to amino residues 83 to 97 of SEQ ID NO:1 (cCAF) or SEQ ID NO:3 (human IL-8), i.e., amino acid sequences encompassing the C-terminal alpha helical domain, may be employed in the practice of the present invention. Similarly, an angiogenic fragment of the present invention can include polypeptides encoded by nucleotide sequences that are at least 70% identical to the portions of SEQ ID NOs 2 and 4 that encode the C-terminal alpha helical domains, e.g., nucleotides 324 to 368 of SEQ ID NO:2 or nucleotides 351 to 392 of SEQ ID NO:4. Preferably the amino acid or nucleotide sequences are at least 80% identical, more preferably 90% identical, and most prefably 95% identical. As defined herein, percent identity is determined using FASTA and BLAST wherein the sequences are aligned so that the highest order match is obtained.

III. Other Biological Activities

Angiogenic ELR-CXC chemokines, such as cCAF and IL-8, are multifunctional proteins. For example, cCAF and IL-8 act as chemoattractants for leukocytes. More particularly, cCAF is chemotactic in vivo for both monocytes/macrophages and lymphocytes, but not heterophils. In vivo administration of cCAF in low doses (100-300 ng/5 μl pellet) is also associated with hyperproliferation of ectodermal cells, elongation and parallel alignment of fibroblasts, and depostition of collagen fibers similar to the granulation tissue of wounds. However, these wound-healing effects are diminished at higher concentrations (˜450-1000 ng/˜3 mm² pellet) of the protein.

Applicant's discovery that a 28 amino acid C-terminal peptide of cCAF has angiogenic properties, points to the alpha helical domain as being responsible for the angiogenic activities of the protein. However, the C-terminal peptide does not have chemotactic properties for leukocytes and does not cause development granulation tissue regardless of the dosage. In accordance with these findings, preferred versions of the present invention utilize angiogenic polypeptides that have angiogenic properties, but are devoid chemotactic properties for leukocytes and do not induce the formation of granulation tissue.

A smaller form of cCAF (˜7 kDa), produced by cleavage at the N-terminus, binds to interstitial collagen and basement membrane components, including laminin and complex proteoglycans, but it does not bind to collagen IV. In addition, the smaller form binds the extracellular matrix (ECM) molecule tenascin, but not fibronectin and hyaloruonic acid. Since this processing step could be relevant to wound healing, preferred versions of the present invention will utilize angiogenic polypeptides that retain the ability to bind such extracellular components.

V. Methods of Making

The preparation of wild type, mutant, native, recombinant and synthetic CXC chemokines will be straightforward to those of skill in the art in light of the present disclosure.

Native cCAF cytokine is secreted as a 9 kDa polypeptide in both normal and transformed cells in culture. The 9 kDa form exists primarly as monomers in the supernatant of cultured CEFs and is stable for a period of 24 hours in normal cultures, but has a half-life of 3 hours in transformed CEFs. cCAF is synthesized and secreted very rapidly, in less than 10 min. The 9E3 is stimulated by a variety of agents, such as oakdaic acid, LPS, and vanadate, with thrombin being the strongest natural activator of this gene so far identified. cCAF can be purified from the supernatant of cultured cells by standard chromatographic methods as previously described (Martins-Green et al., 1998; incorporated herein by reference).

When cells are cultured on extracellular matix molecules, a smaller form of the protein (˜7 kDa) can be produced by post-secretory cleavage at the N-terminus. Plasmin is the only enzyme known to cleave cCAF to yield a C-terminus bearing smaller form of the same size as found when cells are cultured on ECM. (see Martins-Green et al., 1996, incorporated herein by reference).

To prepare a composition comprising a recombinant angiogenic polypeptide all that is required is to express the alpha helical encoding segments of an angiogenic ELR-CXC chemokine gene, including wild type and mutant genes, in a recombinant host cell and to collect the expressed angiogenic polypeptide to obtain the composition. The coding segments can be in the form of naked DNA, or housed within any one of a variety of gene therapy vehicles, such as recombinant viruses or cells, including tumor cells and tumor infiltrating lymphocytes, modified to contain and express the encoded angiogenic polypeptide or peptide.

Synthetic peptides, which contain the C-terminal alpha helical domain of angiogenic ELR-CXC chemokines can be made using automated methods for peptide synthesis. Techniques for the operation of automated peptide synthesizers is standard practice in the art and such services may be obtained commercially, as described further in a subsequent example.

IV. Compositions

Various pharmaceutical compositions and techniques for their preparation and use will be known to those of skill in the art in light of the present disclosure. For a detailed listing of suitable pharmaceutically suitable compositions and associated administrative techniques one may refer to texts such as Remington 's Pharmaceutical Scineces, 18^{th} ed., 1980, Mack Publishing Co., incorporated herein by reference.

Compositions of the present invention contain an effective amount of one or more angiogenic polypeptides derived from ELR-CXC chemokine proteins, polypeptides or peptides. An effective amount is generally an amount sufficient to induce a desired biological activity, such as angiogenesis, migration of endothelial cells, or binding to components of the extracellular matrix.

In preferred embodiments an effective amount of the composition is an amount sufficient to induce a significant increase in angiogenic activity. Most preferably, the significant increase is marked by increased tortuosity of the smaller vessels and/or a marked increase in the sprouting of new blood vessels.

In a clinical context, the effective amount the composition will depend on the host animal or patient, the condition to be treated, and the route of administration. Moreover, the precise amounts of angiogenic polypeptide required will depend on the judgement of the practitioner and may be optimized to the individual by monitoring the biological effects and adjusting the dose accordingly. Although some dosage modification may be necessary, the determination of a suitable dosage range will be straightforward in light of the data presented herein. For example, as disclosed herein, compositions comprising methylcellulose pellets that contained about 450 ng to about 1,000 ng of a 28 amino acid polypeptide, where the total volume of the composition was about 5 μl, were particularly effective in the CAM assay. Accordingly, clinical doses that result in a local concentration of angiogenic peptide of about 30 to 70 μM are contemplated to be particularly useful.

VI. Methods of Use

In certain embodiments, the present invention concerns methods for inducing angiogenesis. These methods generally comprise administering to an animal or a human subject a biologically effective amount of a composition containing a angiogenic fragment of a CXC chemokine. “Angiogenesis” means the process of new blood vessel growth, as may occur in disease states and wound healing. The term “new” describes blood vessels that are present in a number in excess of that observed in the normal state and does not represent the age of any particular vessel within a given individual.

Any consistently observed increase in angiogeneisis in reposnse to the presence of a particular composition is evidence of the induction of angiogenic activity, and establishes the composition as a useful angiogenic composition. However, it will be understood that the most useful agents will result in a significant increase in angiogenesis. “A significant increase in angiogenesis or angiogenic activity” is defined herein as a consistently observed marked increase in the number of new blood vessels, or the establishment of a significant angiogenic state.

Many systems are available for assessing angiogenesis. For example, angiogenesis may be assessed using models of would healing, e.g., in cutaneous or organ wound repair. It may also be assessed by counting microvessels in tissue sections, i.e., determining their density, as described further in the Experiments section below.

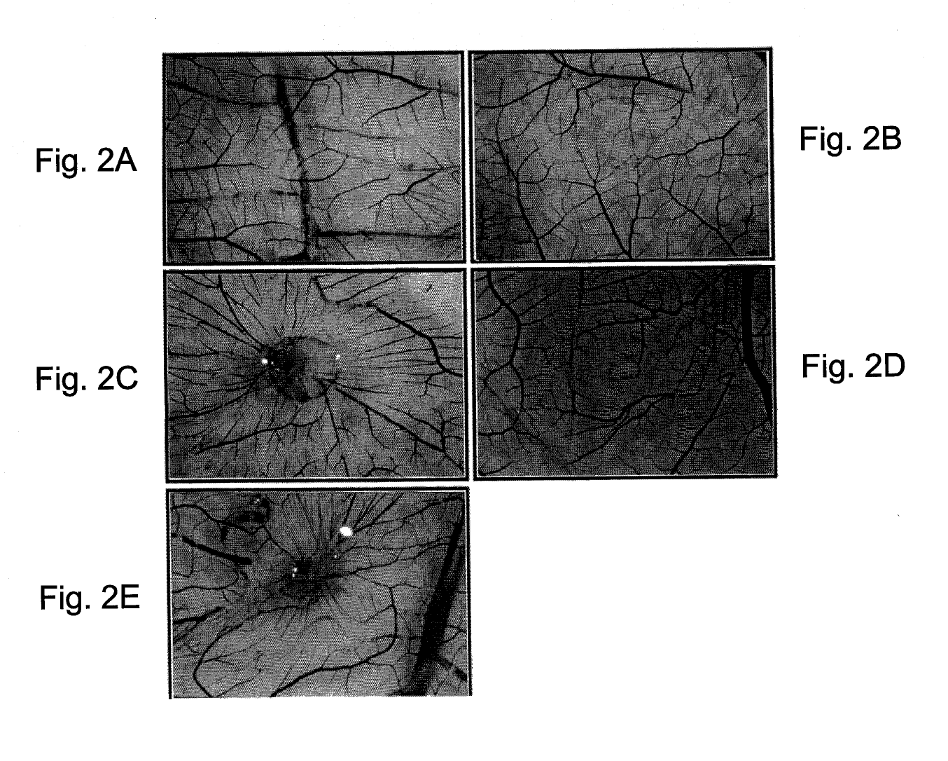

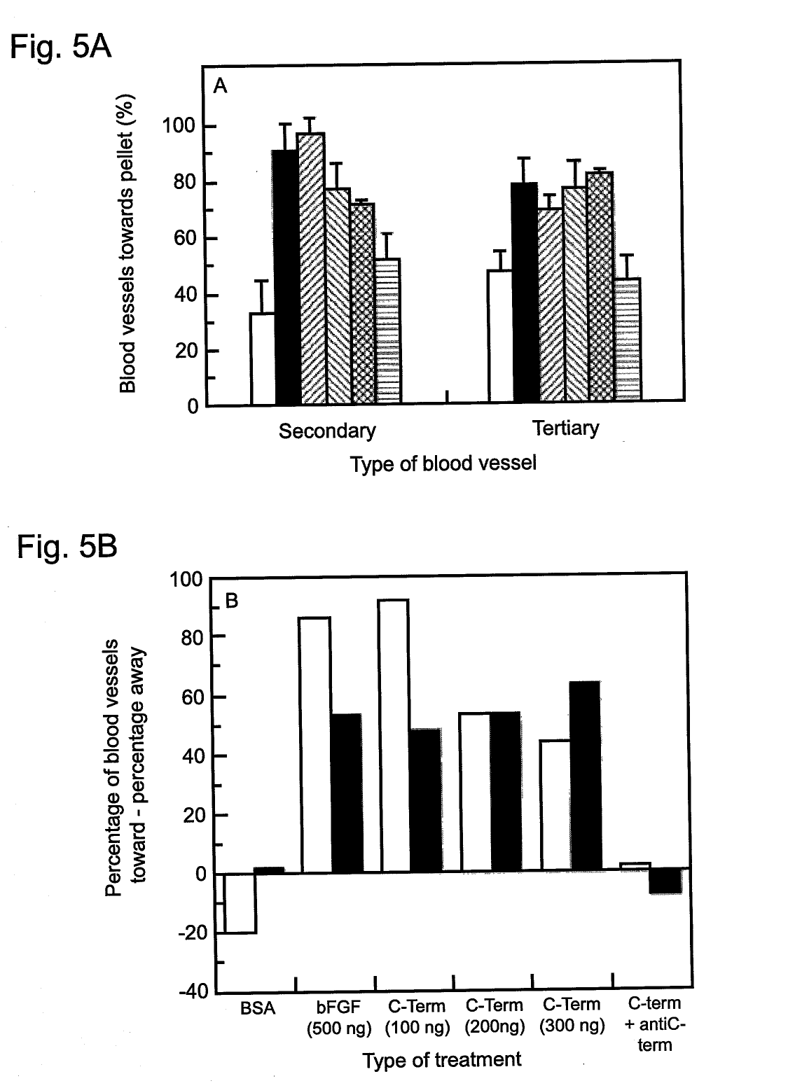

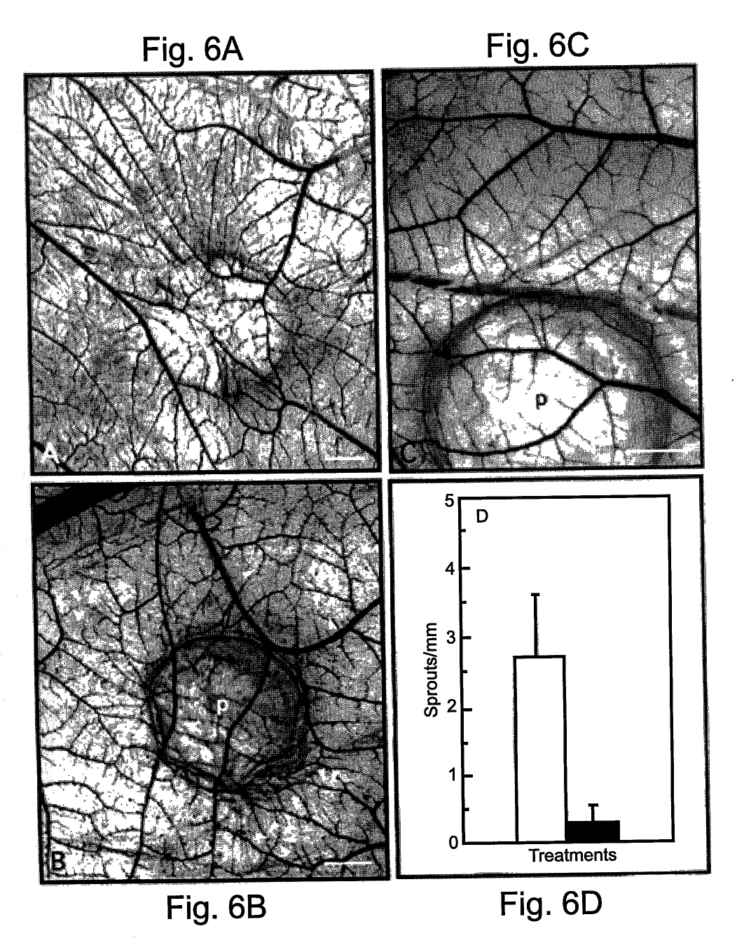

The system most preferred by the present inventor for assessing angiogeneisis is the chorioallantoic membrane (CAM) assay, which is also described in further detail in the Experiments section below. In the CAM assay an angiogenic agent is one that consistently acts to promote the growth of one or more blood vessels upon the CAM, preferably without the evidence of the influx of leukocytes. Localized administration of lower doses (about 100 ng) of an angiogenic agent, namely the C-terminal 28 aa peptide of cCAF, induces reorientation of the secondary vessels (see, e.g., FIG. 2C), whereas higher doses (about 300 ng) are more effective in attracting tertiary blood vessels in the direction of the compositon (see FIG. 5). However, the most effective doses (>450 ng) induce a significant increase in angiogenic activity, marked by less blood vessel reorientation than lower doses, accompanied by increased tortuosity of the smaller vessels and a marked increase in the sprouting of new blood vessels. (See FIG. 6)

Although systemic administration is possible, local or directed administration of the angiogenic composition is preferred, particularly for wound healing. Accordingly, the angiogenic composition may be administered by using direct injection, transdermal techniques or implants; applied in the form of a cream, ointment, gel or lyophilized powder; or may be incorporated into a patch, bandage or wound dressing.

Localized administration of an effective amount of an angiogenic composition, e.g, about 450-1000 ng of polypeptide in about 5 μl of carrier, will advantageously produce a local elevation of the levels of the angiogenic polypeptides. When applied in an area close to a wound, the angiogenic composition can induce sprouting of blood vessels, which can expedite wound healing. In addition, diffusion of the angiogenic polypeptides may result in the formation of a chemotactic and/or haptotactic gradient of angiogenic peptides, which may or may not be bound to the ECM, that promotes migration of endothelial cells and reorientation of secondary blood vessels in the direction of the wound.

In certain embodiments, the present invention concerns methods for inhibiting angiogenesis. These methods generally comprise administering to an animal a biologically effective amount of a composition containing an angiogenesis inhibitor. The angiogenesis inhibitor will typically interfere with binding of the carboxy-terminal region of the angiogenic CXC chemokine to a receptor or the ECM. An example of such an inhibitor would be a neutralizing antibody that binds to the carboxy-terminal region of the angiogenic CXC chemokine, thereby interfering with further intermolecular interactions. Inhibition can be assessed by the angiogenesis assays described above. Alternatively, since angiognesis is required for solid tumor growth, the inhibition of tumor growth in an animal model may be used as an index of the inhibition of angiogenesis.

The invention also has important applications as a bioassay of angiogenesis or angiostasis, i.e. the inhibition of angiogenesis. By providing both positive and negative controls in assays of angiogenesis, the invention may provide control values for comparing the effectiveness of other angiogenic agents or angiogenesis inhibitors.

Although the present invention has been described in considerable detail with reference to certain preferred versions thereof, other versions are possible. For example, it is contemplated that the angiogenic polypeptide of the present invention may be a recombinant fusion protein. Such a fusion protein could include heterologous amino acid sequences from another protein, which provide a second functional domain. Therefore, the spirit and scope of the appended claims should not be limited to the description of the preferred versions described herein.

### EXAMPLES

Synthesis of the C-Terminal Peptide

For these studies, we used the C-terminal 28-aa peptide of cCAF extending from the fourth cysteine. The peptide was prepared as described in Martins-Green et al. (1992). Briefly, the last 28 aas of the C-terminus of the molecule, which includes cysteine No. 4 as the N-terminal most aa, were synthesized by Millegen Biosearch (S. Rafael, Calif.) and purified by HPLC to greater than 90% purity.

Because this peptide contains a cysteine at its N-terminus, it can form dimers under non-reducing conditions by forming disulfide bonds. Immunoblots of the peptide under non-reducing conditions showed a predominance of a 6-kDa band indicating dimer formation, whereas under reducing conditions a 3-kDa band predominated (not shown). Because chemokines can function both as monomers and dimers [see Horuk R (1996) In Horuk R (ed.) Chemoattractant Ligands and Their Receptors, 337 pp. New York: CRC Press], we took advantage of the disulfide bridge formation to determine the effects of both monomers and dimers on angiogenesis by performing experiments in the CAM with the peptide under reducing and non-reducing conditions.

Iodination of the C-Terminal Peptide

The peptide was labeled with ¹²⁵I using the chloramine T method [Greenwood F C, Hunter W M (1963) The preparation of ¹³¹I labeled human growth hormone of high specific activity. Biochem J 89:114-124], modified by substantial reduction of the oxidizing agent to minimize overiodination and damage of the peptide. In brief, 20 μl of 0.5 M phosphate buffer, pH 7.5 and 1.5 μl of Na¹²⁵I were added to the vial containing the dissolved 9E3 peptide. Then 10 μl of chloramine T (400 ng/ml) were added and the tube agitated mildly for 1 min. At the end of this period of time 100 μl of metabisulfite (240 μg/ml) and 300 μl of potassium iodinate (10 ng/ml) were added. The latter two reagents were made up in 0.05 M phosphate buffer, pH 7.5, immediately before iodination. The total content of the vial was then laid over a 10-ml Sephadex G-25 column, previously coated with BSA and eluted in 1-ml aliquots. Most of the iodinated peptide came out in one fraction, followed three fractions later by the nonreacted ¹²⁵I.

Time-Dependent Release of the 9E3 Peptide from Methylcellulose Pellets

Methylcellulose pellets containing either form of the peptide at different concentrations were placed on mature CAMs [see Martins-Green M, Feugate J E (1998) The 9E3/CEF4 gene product is a chemotactic and angiogenic factor that can initiate the wound healing cascade in vivo. Cytokine 10:522-535]. Because the peptide is much smaller than cCAF itself, it is possible that release from the pellet might be much more rapid than for the entire molecule. Therefore, before performing the experiments on the CAM we determined the kinetics of release of the C-terminal peptide from pellets under reducing conditions for which the peptide will be a monomer and therefore of smallest size.

Five microliters of 0.5% methylcellulose (Fisher) containing 300 ng of ¹²⁵I-labeled 9E3 peptide were placed in the bottom of 15 Eppendorf tubes (0.5 ml), allowed to dry and then covered with 300 μl of sterile PBS containing 0.02% sodium azide and incubated at 37° C. Periodically, 280 μl of saline were removed for scintillation counting (20 μl were left behind to ensure that the pellet was not removed) and replaced by fresh saline. The label released into the fluid was counted in a scintillation counter to determine if and over what period of time the peptide was released.

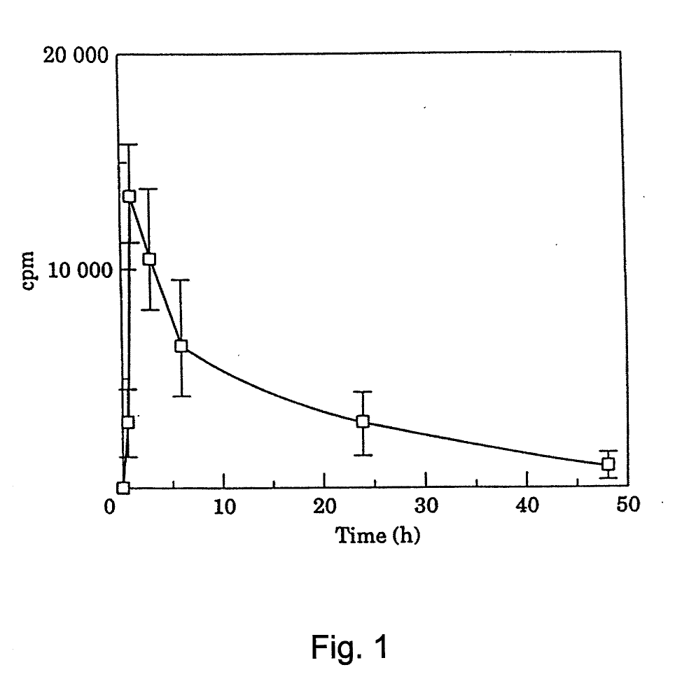

The majority of the peptide was released during the first 6 hours of incubation followed by a slow, prolonged, release up to 48 h (FIG. 1). Therefore, experiments on the CAM were performed for periods of up to 4 days (the same as previously used for the full cCAF molecule) [Martins-Green et al. (1998)].

Angiogenesis Assays in the CAM (Chorioallantoic Membrane)

These assays were done as previously described [Martins-Green et al. (1998)]. Briefly, fertilized chicken eggs were incubated for 3 days at 37° C., at which time 2 ml of albumin were withdrawn to drop the embryo and CAM away from the shelf. Windows were made on day 4, the holes covered with transparent tape, and the eggs placed back in the incubator until day 10 when the experiment was performed. Pellets were prepared by mixing the appropriate amount of reduced and non-reduced 9E3 peptides or bFGF with 1% methylcellulose (1:1) and then delivering 5-μl droplets on a bacterial dish and allowing them to dry in a sterile hood for approximately 30 min. This procedure generated small disc-shaped pellets 2 mm in diameter and ˜0.1 mm thick. These pellets containing various concentrations of protein were placed, under sterile conditions, between two large blood vessels of the 10-day-old CAMs. Two or four days later, the pellets and the surrounding area of the CAM were fixed in situ; the CAMs were then removed and examined for the presence of chemotaxis and/or angiogenesis. Angiogenesis was quantified by analysis of blood vessel orientation and/or increase in the number of vessels and sprouts (see below). Negative control pellets were prepared in the same way with 0.5% methylcellulose alone or with the same concentration of BSA as used for the cCAF peptide or with peptide preincubated with an antibody made against it [Martins-Green et al. (1992)].

Effects of Lower Concentrations of the C-Terminus Peptide

As previously described, 10-day-old CAMs were used because at this stage of development the CAM close to the embryo is fully developed; the blood vessels in this area have stopped growing and assumed a tree-like pattern with short stubby tertiary branches (FIG. 2A). When treatments are applied, changes in this pattern can be attributed to experimental procedures; hence it is possible to test a specific molecule for its angiogenic properties by determining if its presence causes distortion of and/or increase in the number of blood vessels, or if it causes oriented blood vessel growth in comparison with controls. [Martins-Green et al. (1997); Vu M T, Smith C F, Burger P C, Klintworth G K (1985) Methods in laboratory investigation. An evaluation of methods to quantitate the chick chorioallantoic membrane assay in angiogenesis. Lab Invest 4:499-508; Klagsbrun M, D'Amore P A (1991) Regulators or angiogenesis. Annu Rev Physiol 53:217-239; Cockerill G W, Gamble J R, Vadas M A (1995) Angiogenesis: models and modulators. Interntl Rev Cytol 1.59:113-160; and Swerlick R A (1995) Angiogenesis. J Dermatol 22:845-852.]

When compared with natural CAMs (untreated controls; FIG. 2A), CAMs exposed to pellets containing only 0.5% methylcellulose in H₂O or methylcellulose plus bovine serum albumin (BSA) (FIG. 2B) showed no evidence of blood vessel growth or reorientation. In contrast, pellets containing 100-300 ng of the C-terminal peptide (FIG. 2C) stimulated the blood vessels to grow towards the pellet; the effect was eliminated (FIG. 2D) by preincubation of the peptide with excess of an antibody prepared against it [Martins-Green et al. (1992)]. Basic fibroblast growth factor (bFGF) at a dose (500 ng/pellet) previously used by others in the CAM assay [Yang E Y, Moses H L (1990) Transforming growth factor β1-induced changes in cell migration, proliferation, and angiogenesis in the chicken chorioallantoic membrane. J Cell Biol 111:731-741] was used as a positive control (FIG. 2E) because it has been shown in the CAM assay that this growth factor has a direct angiogenic effect on endothelial cells [Saksela O, Moscatelli D, Rifkin D B (1987) The opposing effects of fibroblast growth factor and transforming growth factor beta on the regulation of plasminogen activator activity in capillary endothelial cells. J Cell Biol 105:975-963; and Sato Y. Rifkin D R (1988) Autocrine activities of basic fibroblast growth factor: Regulation of endothelial cell movement, plasminogen activator synthesis, and DNA synthesis. J Cell Biol 107:1199-1205]. This dose of bFGF is equimolar to 100 ng or C-terminal peptide.

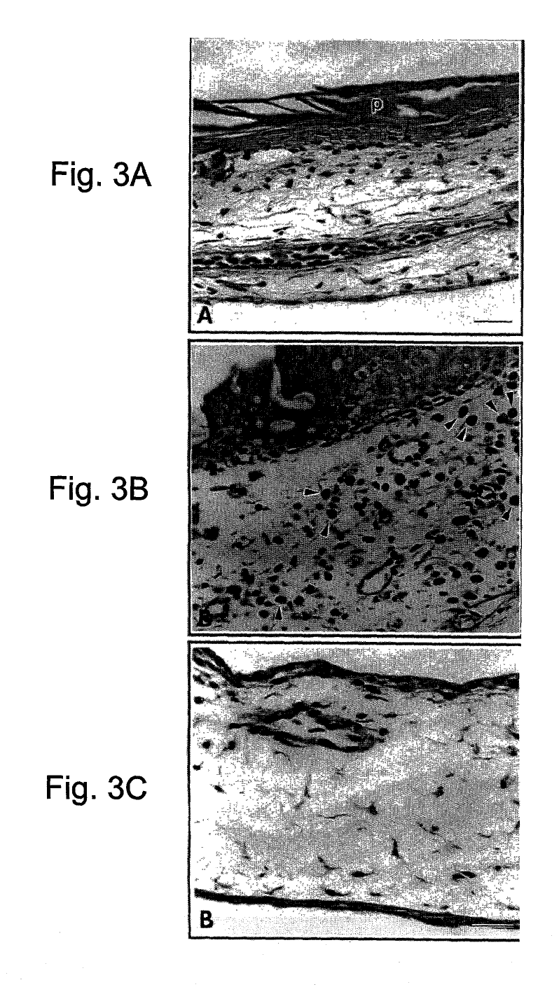

Cross sections of CAMs examined two days after application of pellets containing the peptide showed no chemotaxis for leukocytes (FIG. 3A), in contrast to the whole cCAF molecule which at the same concentrations is chemotactic for monocyte/macrophages and lymphocytes (FIG. 3B). FIG. 3C shows a cross section through a normal CAM for comparison. Although the general structure of treated and untreated CAMs is the same, some differences were seen. In particular, the ectoderm of the CAM treated with the peptide appears slightly hyperproliferated. This was also observed, but to a much larger extent, with the full molecule (FIG. 3B; see also Martins-Green et al., 1998).

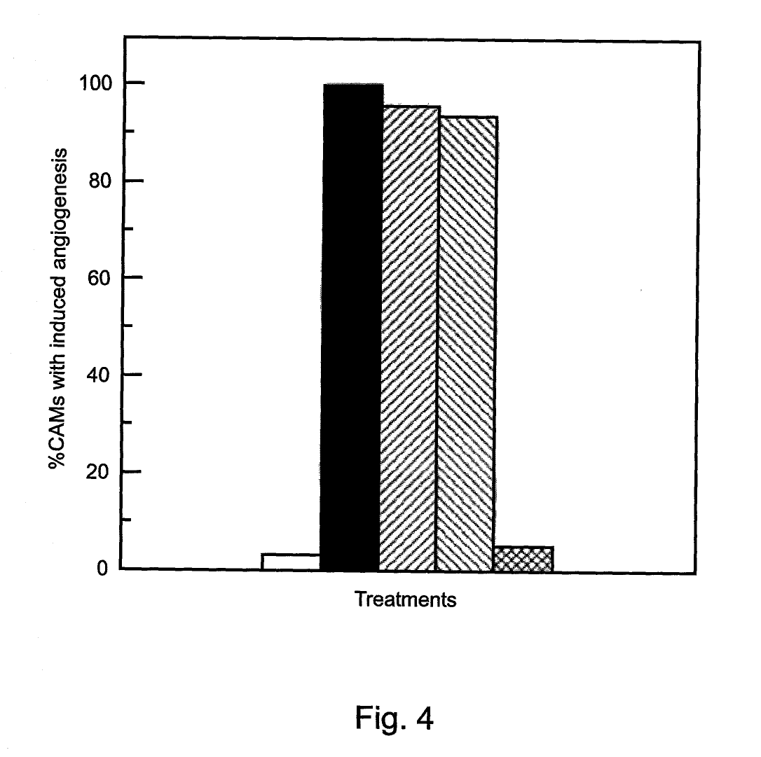

For each treatment described above, we determined the percentage of CAMs showing reorientation and/or growth of blood vessels towards the pellet (FIG. 4). In control pellets containing BSA, 1 of 31 (3.2%) showed distortion of the blood vessels in its vicinity, whereas 46 of 48 pellets (96%) containing the C-terminal peptide under reducing conditions induced oriented blood vessel growth. Experiments performed with pellets containing non-reduced peptide showed that 15 out of 16 pellets (94%) stimulated oriented blood vessel growth. Very similar effects were observed in 6 out of 6 (100%) CAMs treated with pellets containing bFGF.

Quantification of Angiogenesis

The CAMs were then analyzed by making measurements of the orientation of the blood vessels relative to the pellet. For each concentration of the C-terminal peptide and for all controls, 6-10 CAMs were measured. Vessels were classified (FIG. 2A) as primary (largest), secondary (branches of primary vessels) and tertiary (all other vessels). In all cases, the orientations of blood vessels in areas comparable to those shown in FIG. 2 were determined and classified as toward or away from the pellet by hand. The measurements were made without prior knowledge of which specimens were controls and which were cCAF. For secondary vessels; we looked for deviations in their orientations. For tertiary vessels, each vessel was approximated by a vector pointing in its growth direction. Each vector was resolved into components directed radially and tangentially with respect to the pellet; the definition of toward or away was determined from the radial component of the vector. For graphical presentation of the data, the percent of blood vessels growing toward the pellet was determined for secondary and tertiary vessels (FIG. 5).

Negative controls (BSA and C-terminal peptide+anti-C-terminal peptide) showed essentially random blood vessel orientation (approximately 50% of both secondary and tertiary blood vessels registered toward and away from the pellet), whereas the positive control, bFGF (500 ng), showed over 90% of the secondary vessels deviated toward the pellet and slightly less than 80% of the tertiary vessels pointing in the direction of the pellet (FIG. 5A).

Comparison of these results with those obtained with the C-terminal peptide at three different concentrations (100 ng, 200 ng, 300 ng), showed that the lower concentration of the peptide was more effective in stimulating deviation of the secondary blood vessels towards the pellet and the higher concentrations more effective for the tertiary vessels. In both cases, application of the Student t-test to the values at 100 ng and 300 ng peptide yielded differences that are significant (FIG. 5A).

To better illustrate the significance of these differences, the data were regrouped and plotted as the percent of blood vessels oriented towards the pellet minus the percent directed away from the pellet (FIG. 5B), thus essentially subtracting out the randomly oriented vessels. Therefore, negative controls, with little preferred orientation registered values close to zero, whereas bFGF and all cCAF peptide concentrations (100-300 ng) showed marked blood vessel orientation toward the pellet. This plot shows particularly clearly that increasing doses of the peptide (up to 300 ng) resulted in progressive decline of the deviation effect on the secondary vessels but also resulted in a systematic increase in alignment of tertiary vessels towards the pellet.

High Doses (450-500 ng) of cCAF and IL-8

CAMs were treated with pellets containing 450 ng of cCAF or 500 ng of IL-8, essentially as described above. At 4 days after application of the pellets, both cCAF and IL-8 caused extensive new sprouting of blood vesselsof the CAM, whereas BSA controls showed no new sprouting (data not shown).

Effects of Higher Concentrations of the C-Terminal Peptide

When higher doses of the C-terminal peptide were applied (600-1000 ng/pellet), reorientation of blood vessels was greatly decreased (compare FIG. 6A with FIG. 2C), but they exhibited marked tortuosity (compare FIGS. 6B and C) and showed abundant sprouting. These effects were eliminated (not shown, but see FIG. 2D) by preincubation of the peptide with excess of an antibody directed against it [Martins-Green et al. (1992)].

Quantification of blood vessel sprouting was performed as described previously [Martins-Green et al. (1998)]. Briefly, a sprout was defined as a very small blood vessel having a length of one third or less that of the tertiary branches of the normal CAM. Sprouts thus defined were counted in the areas of the CAMs with pellets containing 1000 ng of C-terminal peptide. For CAMs treated with 1000 ng/pellet of C-terminal peptide, we counted sprouts on 90 mm total of 21 individual blood vessel segments from 6 different CAMs. For the BSA controls we counted sprouts on 123 mm of 24 segments of blood vessels. FIG. 6D shows that the number of new sprouts on the CAMs treated with the C-terminal peptide was about 10 times greater than that found in the CAMs treated with BSA.

Angiogenic Assays in 2-Week-Old Chicks

To further test the angiogenic potential of the C-terminal peptide, 1 μg of the peptide in 10 μl of water was deposited under the skin of the underside of the wing of 2-week-old chicks on two consecutive days, and the tissues prepared for histology on the fourth day.

The C-terminal peptide was deposited under the skin of the under wing of 1-week-old chicks using a procedure described previously [Martins-Green M, Boudreau N, Bissell M J (1994) Inflammation is responsible for the development of wound tumors in RSV-infected newly-hatched chicks. Cancer Res 54:4334-4341]. Two samples of 1 μg/10 μl each, were applied on two consecutive days followed by a waiting period of 3 days. The wings were collected and prepared for histology as described previously [Martins-Green et al.(1994)]. Sections were cut and stained with eosin and haematoxylin and observed for the presence of new blood vessel growth in the area of peptide application.

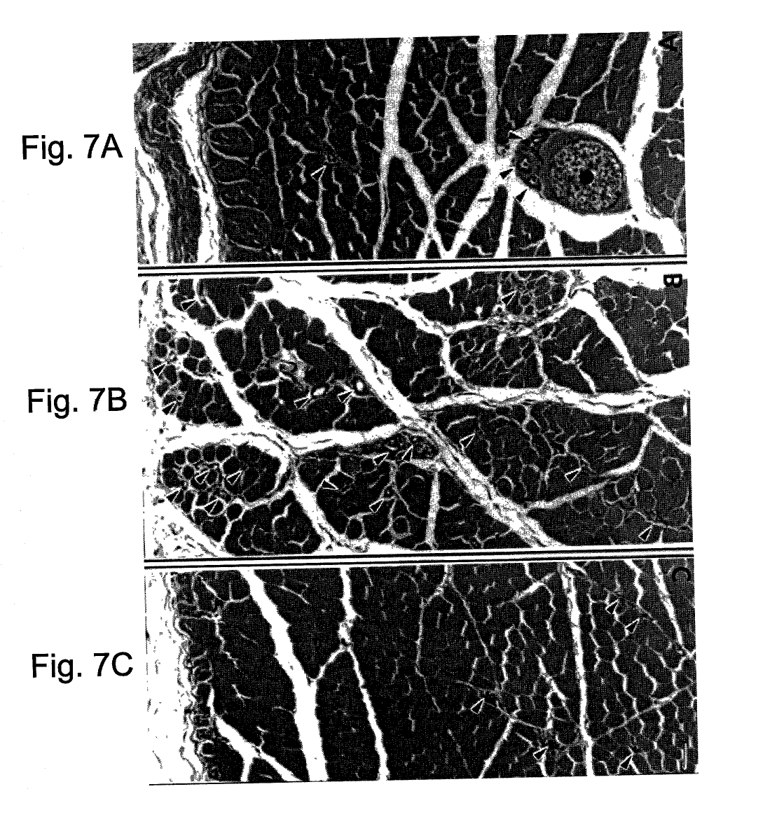

In wing cross-sections, the skin lies directly on top of the muscle blocks which are composed of many muscle fibers (FIG. 7). Each muscle fiber is surrounded by a basement membrane and is separated from its neighbors by a thin layer of connective tissue, which contains very sparse, small, blood vessels that provide the blood supply to the muscle fibers (FIG. 7A). Four days after the first dose of peptide, the muscle tissue underlying the site of application had many more small blood vessels (FIG. 7B) than did either the control to which vehicle alone had been applied (FIG. 7C) or the normal wing (FIG. 7A).

Discussion

The studies presented here show that the C-terminus (28 aa) of the cCAF chemokineis angiogenic in vivo. At low concentrations (100-300 ng/pellet) it causes oriented blood vessel growth of the secondary and tertiary vessels of the CAM. At higher concentrations it causes tortuosity of the existing tertiary blood vessels and stimulates their sprouting. Furthermore, when applied under the skin of the wing of newly hatched chicks it causes an increase in the number of microvessels in the connective tissue surrounding the muscle fibers. However, in contrast to previous results with the full cCAF molecule [Martins-Green et al. (1998)], no evidence was found for leukocyte chemotaxis or granulation-like tissue formation.

As was found earlier for the entire protein [Martins-Green et al. (1998)], the angiogenic effects of the peptide are dose dependent. At lower doses (100-300 ng), the peptide causes oriented blood vessel growth but it affects the secondary and tertiary blood vessels differentially; the most effective dose for secondaries is ˜100 ng and for the tertiaries is ˜300 ng. In the case of the former, the pre-existing vessels are diverted locally toward the pellet whereas in the latter the vessels are greatly extended and grow directly toward the pellet. One possible explanation for these differences is that the endothelial cells of secondary and tertiary blood vessels contain receptors with different affinity for the peptide: Higher affinity receptors in the secondaries and lower affinity in the tertiaries. If this is the case, at doses of ˜300 ng the peptide could cause desensitization of the receptors in the secondary blood vessels and abolish the chemotactic response. Contrarily, low affinity receptors in the tertiary blood vessels would require higher concentration of ligand to be activated and cause oriented blood vessel growth. In support of this possibility is the observation that at a dose of 200 ng, the effects on both sizes of vessels is equal. This type of desensitization of cells has been observed in response to high doses of other chemokines [Baggiolini et al. (1994); Horuk; and Rutledge B J, Rayburn H, Rosenberg R, North R J, Gladue R P, Corless C L. Rollins B J (1995) High level MCP-1 expression in transgenic inice increases their susceptibility to intracellular pathogens. J Immunol 155:4838-4843]. Indeed, chemokines are ligands for seven transmembrane receptors and these receptors in general are desensitized by excess ligand (e.g. Strosberg A D (1995) Structure function and regulation or the three beta-adrenergic receptors. Obesity Res 3 (Suppl. 4):501 S-505 S; Hipkin R W, Friedman J, Clark R B, Eppler C M, Schonbrunn A (1997) Agonist-induced desensitization, internalization, and phosphorylation of the sst2A somatostatin receptor. J Biol Chem 272:13869-13876; and Hipkin R W, Friedman J, Clark R B, Eppler C M, Schonbrunn A (1997) Agonist-induced desensitization, internalization, and phosphorylation of the sst2A somatostatin receptor. J Biol Chem 272:13869-13876).

At the highest doses tested (600-1000 ng/pellet), the peptide (and the entire cCAF molecule) [Martins-Green et al. (1998)] causes less blood vessel orientation toward the pellet, but stimulates pronounced tortuosity and extensive sprouting of the tertiary blood vessels. The lack of directionality of blood vessels around the pellet could be due to the loss of the chemotactic gradient and therefore the blood vessels behave as if the area has been flooded with the molecule. This interpretation is supported by the work of Thompson et al. [Thompson W D, Campbell R, Evans T (1985) Fibrin degradation and angiogenesis: Quantitative analysis of the angiogenic response in the chick chorioallantoic membrane. J Pathology 145:27-37] who observed increased vascularity and tortuosity when CAMs were flooded with fibrin fragments. It is known that sprouting in general is preceded by an increase in vascular permeability and vasodilation which reflect loss of the integrity of the basement membrane and allow migration of endothelial cells that form the sprout [Clark et al.; Swerlick; Saksela et al.; Sato et al.; Sholley M M, Ferguson G P, Seibel H R, Montour J L. Wilson J D (1984) Mechanisms of neovascularization: Vascular sprouting can occur without proliferation of endothelial cells. Lab Invest 51:624-634; Folkman J, Sing Y (1992) Control of angiogenesis by heparin and other sulfated polysaccharides. Adv Exp Med Biol 313:355-364; Wijelath E S, Carlsen B, Cole T, Chen J, Kothari S, Hammond W P (1997) Oncostatin M induces basic fibroblast growth factor expression in endothelial cells and promotes endothelial cell proliferation, migration and spindle morphology. J Cell Sci 110:871-879; and Wijelath E S, Carlsen B, Cole T, Chen J, Kothari S, Hammond W P (1997) Oncostatin M induces basic fibroblast growth factor expression in endothelial cells and promotes endothelial cell proliferation, migration and spindle morphology. J Cell Sci 110:871-879]. Such loss of integrity of basement membrane and local migration of endothelial cells might be responsible for the tortuosity observed here and by Thompson et al. and the correlation of tortuosity and sprouting observed for both the C-terminal peptide and the entire cCAF molecule [Martins-Green et al. (1998)].

A variety of studies have shown that the sequence of three amino acids, ELR, constitutes a motif present in the N-terminus of the C—X—C chemokines that is important for their chemotaxis for neutrophils [Clark-Lewis I, Schumacher C, Baggiolini M. Moser B (1991) Structure-activity relationships of Interleukin-8 determined using chemical analogs. Critical role of NH₂-terminal residues and evidence for uncoupling of neutrophil chemotaxis, exocytosis, and receptor binding activities. J Biol Chem 266:23128-23134; and Clark-Lewis I, Dewald B, Geiser T, Moser B, Baggiolini M (1993) Platelet factor 4 binds to interteukin-8 receptors and activates neutrophils when the N-terminus is modified with Glu-Leu-Arg. Proc Natl Acad Sci USA 90:3574-3577]. In IL-8, this same set of residues is crucial for receptor binding to neutrophils [Hébert C A, Vitangcol R V, Baker J B (1991) Scanning mutagenesis of interieukin-8 identifies a cluster of residues required for receptor binding. J Biol Chem 266:18989-18994; and Hébert, Lowman (1996) In Horuk R led.) Chemoattractant Ligands and Their Receptors, 377 pp. New York: CRC Press]. In contrast, the active domain for angiogenesis has not yet been positively identified for any chemokine, although it has been suggested that the ELR motif also is involved in angiogenesis because, of all of the C—X—C chemokines that have been investigated to date, those that contain the ELR motif are angiogenic whereas those that do not contain this motif are not angiogenic. [Strieter et al. (1995a); and Strieter R M, Polverini P J, Arenberg D A, Walz A, Opdenakker G, Van Damme J, Kunkel S L (1995b) Role of C—X—C chemokines as regulators of angiogenesis in lung cancer. J Leukocyte Biol 57:752-762] It is not yet clear if the ELR motif (located in cCAF's N-terminus) is important for cCAF's chemotactic properties for monocyte/macrophages and lymphocytes, but it does appear that this sequence of amino acids is not necessary for the angiogenic properties of this chicken chemokine. As shown here, the C-terminal 28-aa peptide of cCAF (which does not contain the ELR motif) is by itself angiogenic in the CAM. This peptide stimulates both oriented blood vessel growth and sprouting at approximately the same efficiency as the whole protein. These results further suggest that the C-terminus exerts its angiogenic properties directly because changes in the CAMs containing the peptide (other than blood vessel reorientation) were not observed. In particular, chemotaxis did not occur for monocyte/macrophages or any other cell type known to release molecular mediators of angiogenesis. However, these results do not preclude the possibility that other portions of the cCAF molecule are also angiogenic.

Localizing a biological activity to the C-terminal peptide of cCAF would not be a unique phenomenon; C-terminal peptides of other chemokines also have been shown to be functional. For example, the tridecapeptide of PL-4 is angiostatic (Maione et al.) and the C-terminal α-helix of MGSA specifically binds to and is mitogenic for melanoma cells [Roby P, Page M (1995) Cell-binding and growth-stimulating activities of the C-terminal part of human MGSA/Groα. Biochem Biophys Res Commun 206:792-798; and Richmond A. Shattuck R L (1996) Melanoma growth stimulatory activity: physiology, biology, structure/function, and role in disease. In Horuk R (ed.) Chemoattractant Ligands and Their Receptors. 87-124, New York: CRC Press].

Determination of angiogenic properties for the C-terminal 28-aa peptide of cCAF is potentially very relevant physiologically because after secretion this chemokine can be processed at the N-terminus by plasmin resulting in a smaller form (˜7 kDa) that still contains the C-terminus. The smaller form binds to specific extracellular matrix molecules [Martins-Green et al. (1996)], which could favor a conformational change such that the angiogenic determinants are maximally presented to their receptors. Alternatively (or additionally), binding to matrix could cause the establishment of an hapotactic gradient which would favor endothelial cell migration. In newly hatched chicks, such a gradient of cCAF develops during wound healing; the granulation tissue expresses high levels of 9E3/cCAF, and expression falls off progressively with distance [Martins-Green et al. (1992); and Martins-Green et al. (1990)] The appearance of plasmin at the wound site to dissolve the blood clot coincides in time with the initiation of the binding of cCAF to ECM. This binding of the smaller form (which contains the C-terminal peptide used in these studies) to the ECM and production of a persistent gradient of this chemokine might form the basis for the chemotaxis of endothelial cells. Upon arrival of microvessels at the wound site, the locally elevated level of cCAF could induce sprouting of blood vessels. This scenario depicts a biologically very efficient situation, because sprouting would only be induced locally where it is needed.

Although the present invention has been described in considerable detail with reference to certain preferred versions thereof, other versions are possible. Therefore, the spirit and scope of the appended claims should not be limited to the description of the preferred versions described herein.

What is claimed is:

1. An angiogenic composition comprising;
an effective amount of an angiogenic polypeptide, wherein said polypeptide is a C-terminal fragment of a CXC chemokine, said fragment being devoid of chemotactic activity for leukocytes, wherein the effective amount is an amount sufficient to induce tortuosity and sprouting of new blood vessels; and
a pharmaceutically suitable carrier.

2. A composition according to claim 1, the angiogenic polypeptide comprising an amino acid sequence substantially equivalent to a C-terminal alpha helical domain of a wild-type CXC chemokine, wherein said wild-type chemokine contains four conserved cysteine residues, an ELR motif and a WVQ motif, the C-terminal alpha helical domain comprising at least the WVQ motif and continuing C-terminally at least about twelve residues beyond the WVQ motif of the wild-type chemokine sequence, the N-terminus of the polypeptide beginning at a residue downstream from the ELR motif.

3. A composition according to claim 1, the angiogenic polypeptide having an alpha helical domain, wherein the amino acid sequence of alpha helical domain is at least 70% identical to amino acids 83 to 97 of SEQ ID NO: 1 or SEQ ID NO:3 as determined by FASTA or BLAST wherein the sequences are aligned so that the highest order match is obtained.

4. A composition according to claim 2, wherein the alpha helical domain of the angiogenic polypeptide is encoded by a nucleotide sequence which is at least 70% identical to nucleotides 324 to 368 of SEQ ID NO:2 or nucleotides 351 to 392 of SEQ ID NO:4 as determined by FASTA or BLAST wherein the sequences are aligned so that the highest order match is obtained.

5. A composition according to claim 1, wherein the CXC chemokine is selected from the group consisting of cCAF and IL-8.

6. The composition of claim 5, wherein said IL-8 includes human, murine, bovine, pig, rabbit, sheep and other mammalian IL-8 molecules.

7. The composition of claim 1 wherein the angiogenic polypeptide corresponds to amino acid residues 76 to 103 of SEQ ID NO:1.

8. A method for inducing angiogenesis comprising administering to an animal or a human subject a biologically effective amount of the composition of claim 1.

9. A method to improve wound healing comprising administering the composition of claim 1 in proximity to a wound.

10. A method for increasing the density of microvessels in a tissue comprising administering the composition of claim 1 to the tissue.


SEQUENCE LISTING (6 sequences)

SEQ ID NO: 1 - protein, 103 aa, Gallusgallus
MNGKLGAVLALLLVSAALSQGRTLVKMGNELRCQCISTHSKFIHPKSIQDVKLTPSGPHCKNVEIIATLKDGREVCLDPTAPWVQLIVKALMAKAQLNSDAPL

SEQ ID NO: 2 - DNA, 1182 bp, Gallusgallus
  Unsure (1)..(2): n is unsure
nntcagcaatcctctgacaggagagatcacagctccacaaaacctcagctcagaaaacaagccaaacactcctaaccatgaacggcaagcttggagctgtcctggccctcctcctggtttcagctgctctgtcgcaaggtaggacgctggtaaagatggggaatgagctgcggtgccagtgcattagcactcattctaagttcatccaccctaaatccattcaagatgtgaagctgacgccaagcggcccccactgcaagaatgttgaaatcatagctactctaaaggatggaagagaggtgtgcttggaccccactgctccctgggtacagctgatcgtaaaggcacttatggccaaggctcagctcaattctgatgcaccactgtgagaaaattccagacaggaaaaatcctcagaactgctcctgatttctactgggagaaacatccgaagaaggcatcatgaagcattccatcttccaccttccacatcggtgcctcatgttaattgcagatccttgtatctatttatttatttatttaactgcatgtatttaaaaaagtctttcataatggtcagtgctgtgggattcactgtccagtgaaactgaagacactgaatagcaaaagggcttgctaggggaaatgaagatcccttggaagccacttcagtcagacacaatcagttaagtgcaatgcacttacagcacagcttgtttgtattaagccctactgtgttgctattacagcagcaaactggtaattcctcctgctcccctggagtgctctagtatgttgtgtcaacaacagtttcctagtcagagtcagctcatgccgactgcagactgtgtttaaaacttcagaaatctaacctgcagaatctgtaagactgtgggtttggtatttattatgatttccatggtatttataaatatatttatttactagtttctatacaagatggaaggagatgataacttgtgtaatttctactggattttctgttcttaatgatgaatacttaagaaacattcacatacccattactctgcataaggacttggttctatgtctaatacgtgagttattcagctaatggaaaaaaaactacagcatgcatacacagaatttgcttgtgagaatgtaattacctcttacaatatattaataaatattttattt

SEQ ID NO: 3 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 4 - DNA, 1560 bp, Homo sapiens
ctccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacttttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagatatacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaaaaaaaaccgg

SEQ ID NO: 5 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 6 - DNA, 1103 bp, Homo sapiens
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatag